(19) United States
(12) Patent Application Publication
(10) Pub. No.: US 2014/0371688 A1
(43) Pub. Date: Dec. 18, 2014

(54) OPHTHALMIC DEVICE TO GUIDE AND AID THE USE OF AN EYE DROP DISPENSER

(71) Applicant: Nima REZAEI ABBASSI, Syracuse, NY (US)

(72) Inventor: Nima REZAEI ABBASSI, Syracuse, NY (US)

(21) Appl. No.: 13/918,629

(22) Filed: Jun. 14, 2013

(51) Int. Cl. A61F 9/00

(52) U.S. Cl. CPC A61F 9/0026
USPC 604/290; 604/302

(57) ABSTRACT

The present invention relates to an ophthalmic device designed to ease the use of eye drop dispensers. It is one ophthalmic device but can be thought of as consisting of two parts: the nose bridge (11), which rests on the user's nose and the body (19), which connects to the eye drop dispenser. The body is flat and comes off of the nose bridge at an angle, allowing the eye drop dispenser to point toward the eye. The body attaches to the eye drop dispenser by Velcro (18), adhesive tape, strap, or any other measure to adhere two things together. This allows the ophthalmic device to work with any type of eye drop dispenser. Together, the nose bridge and the body, allow the user to securely and easily administer an eye drop to the medial part of their eye.

### BACKGROUND OF THE INVENTION

1. Field of Invention

The present invention relates to an ophthalmic device designed to ease the use of eye drop dispensers.

2. Prior Art

Using eye drop dispensers are difficult for many people. For decades people have tried to design. an easier way for people to self administer eye drops. Numerous patents have been filed for eye drop aids, guides, ophthalmic devices, or apparatus. For example U.S. Pat. Nos. 5,976,116, 4,257,417, 3,934,590, 7,235,065, 8,206,362, 2,722,216, 6,736,802, 4,134,403, 5,713,495, 2,676,592, 4,344,430, 6,595,970, and 8,348,912. However, only a handful of these patents have become commercially available products, and out of those that are commercially available, none have become widely used. Some of the reasons for this are that the ophthalmic devices cannot be used with all types of eye drop dispensers, do not stabilize the user's hand, or do not consistently get the drop into the user's eye.

### BRIEF SUMMARY OF THE INVENTION

The present invention avoids the difficulties encountered in the prior art. The advantage of the present ophthalmic device is that it correctly positions the eye drop dispenser, stabilizes the user's hand, and can be used with any type of eye drop dispenser. The advantage of the present ophthalmic device comes from its design.

It is one ophthalmic device but can be thought of as consisting two parts: the nose bridge, which rests on the user's nose, and the body, which connects to the eye drop dispenser. The nose bridge is arcuate shaped and securely rests on the user's nose, aligns the eye drop dispenser with the user's eye, and allows the user to rest their hand on their forehead for added stability. The body is that and comes off of the nose bridge at an angle, allowing the eye drop dispenser to point toward the eye. The body attaches to the eye drop dispenser by Velcro, adhesive tape, strap, or any other measure to adhere two things together. This allows the ophthalmic device to work with any type of eye drop dispenser. Together, the nose bridge and the body, allow the user to securely and easily administer an eye drop to the medial part of their eye. The advantage of having the eye drop fall in this location is that whether the eye is open or closed, or the eye drop is not perfectly aligned with the eye, the user can still get the eye drop into their eye.

### BRIEF DESCRIPTION OF THE DRAWINGS

The drawings are designed for the purpose of illustration only and not as a definition of the limits of the invention.

FIG. 1 is a perspective view of a ophthalmic device constructed in accordance with the present invention.

FIG. 2 is a top view of the ophthalmic device illustrated in FIG. 1.

FIG. 3 is a bottom view of the ophthalmic device illustrated in FIG. 1.

FIG. 4 is a side view of the ophthalmic device illustrated in FIG. 1.

FIG. 5 is a perspective view of the ophthalmic device illustrated in FIG. 1 attached to an eye drop dispenser.

FIG. 6 is a perspective vim of the ophthalmic device illustrated in FIG. 1 in use.

### DETAILED DESCRIPTION OF THE PREFERRED EMBODIMENT

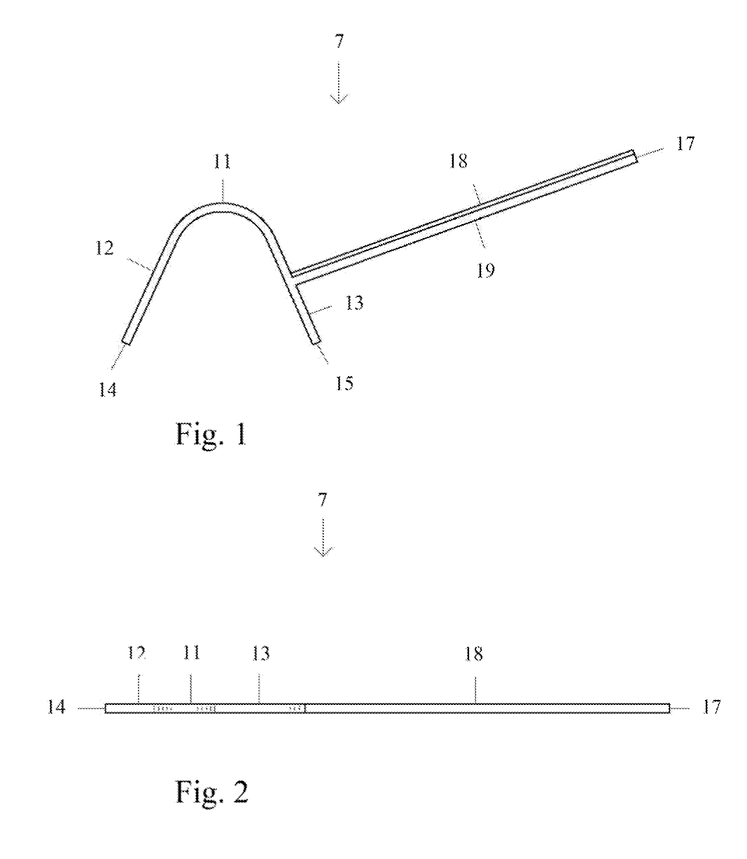

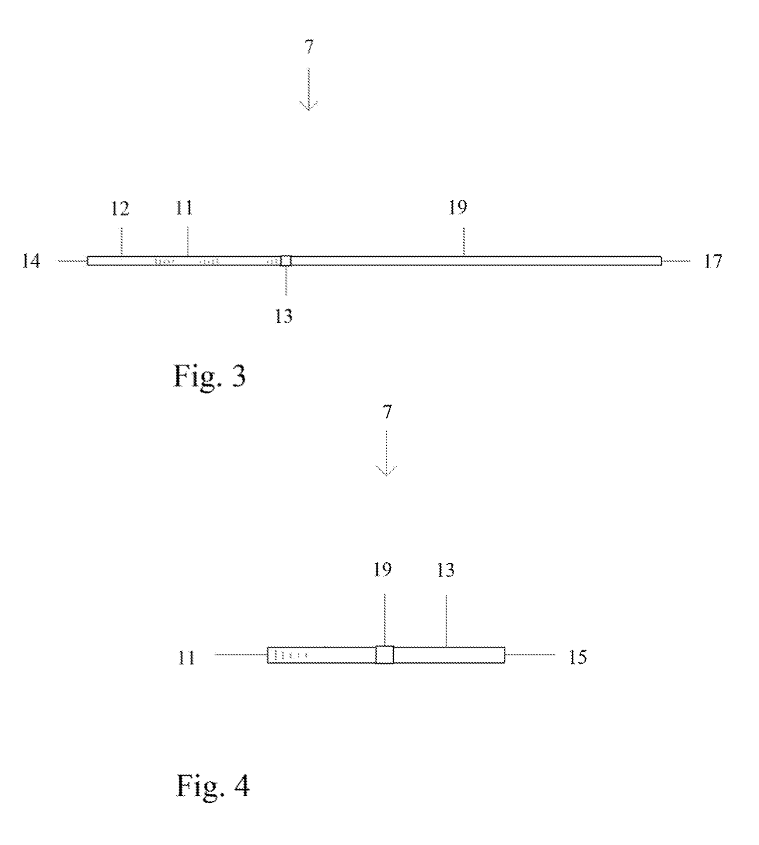

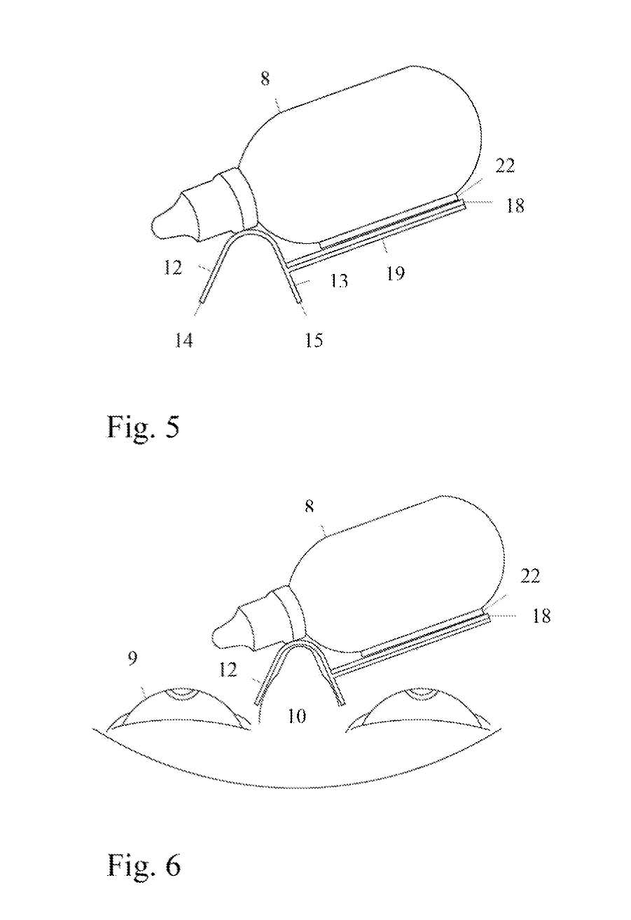

It is to be understood by one of ordinary skill in the art that the present discussion is a description of preferred embodiment only and is not intended as limiting the broader aspects of the present invention. FIGS. 1 through 4 illustrate the preferred embodiment of an ophthalmic device 7 in accordance with the principles of the present invention. Whereas, FIGS. 5 to 6 illustrate how the ophthalmic device 7 is used by a user of the present invention. FIG. 5 shows the ophthalmic device 7 attached to a conventional eye drop dispenser 8. FIG. 6 shows the ophthalmic device 7 attached to a conventional eye drop dispenser 8 over a recipient eye 9 and supported on a facial area 10. None of the figures portray the users hand given it is common knowledge how eye drops are dispensed.

Ophthalmic device 7 has a nasal bridge 11 to assist in correct position and stabilization of the ophthalmic device 7. The nasal bridge 11 is arcuate shaped. A proximal surface 12 is located on the same side as the eye drop dispenser tip and hence the same side of the eye 9 receiving the eye drop. A distal surface 13 is located on the opposite eye, the eye not receiving the eye drop. Both the proximal 12 and the distal surface 13 are flat, the same length, and both the proximal tip 14 and distal tip 15 is designed to be safe to the eye 9.

The distal surface of the nose bridge 13 is attached to a body 15, which serves to receive the eye drop dispenser 8. The body 16 forms an angle of approximately 20 degrees with the nasal bridge 11. The body 16 is flat and slightly longer then the proximal 12 and distal 13 surfaces of the nasal bridge 11; however its tip 17 is similar to their tips. The body 16 has a top surface 18 and a bottom surface 19. The top surface 18 has attached to it a strip of rough Velcro 20, which spans the entire length of the body 16, whereas the bottom surface is bare 21.

The velcro serves to connect the eye drop dispenser 8, which has soft Velcro attached to it 22, to the ophthalmic device 7. This method of connection allows any dispenser type to be used with the ophthalmic device 7. The correct positioning of the eye drop dispenser 8 with the ophthalmic device 7 is achieved by aligning the tip of the eye drop dispenser 8 with the tip of the proximal surface 14 of the nose bridge 11. Once proper alignment has been achieved, the user places the ophthalmic device over the bridge of the nose 10 and squeezes a drop into the medial aspect of the eye. Placing the eye drop in the medial aspect of the eye allows the eye drop to consistently get into the users eye. In addition when using the ophthalmic device 7 the user may rest their hand on their face for added stability (not portrayed in figures) allowing people with tremors to easily use the ophthalmic device 7.

The ophthalmic device 7 can be made from a mold using resilient material such as plastic. The Velcro can then be attached to the ophthalmic device after it has been molded from plastic. In addition Velcro will have to be attached to the eye drop dispenser in order to attach the dispenser to the ophthalmic device. However, while this is preferred embodiment of the invention, modifications and variations may be made by those of skills in the art without departing from the spirit and scope of the present invention. It should also be understood that aspects of various embodiments may be interchanged in whole or in part and the description is an example only, and is not intended to be limitative of the invention described herein.

What is claimed is:

1. An ophthalmic device for facilitating dispensing of an eye drop from an eye drop dispenser, the ophthalmic device comprising:
a) a nose bridge adapted for placement on the user's nose, said nose bridge having an arcuate shape.
b) said nose bridge having a proximal surface and a distal surface, said surfaces are about the same dimension.
c) a body which is attached to the said distal surface of the said nose bridge at an angle.
d) said body is longer than said proximal and distal surface of said nose bridge
e) said body has a top surface and bottom surface
f) said top surface of said body has means for adhering to an eve drop dispenser spanning the entire length of said top surface
wherein the tip of the said proximal and distal surface of the said nose bridge is designed to have blunt edges.

2. The ophthalmic device of claim 1, wherein the said means for adhering an eye drop dispenser to said top surface of said body comprise of Velcro, tape, glue, magnet, rubber band, or any other means of adhering two different objects together.

3. The ophthalmic device of claim 1, whereas the ophthalmic device is in one piece.

4. The ophthalmic device of claim 1, whereas the ophthalmic device is made from plastic material in a single injection mold step.

5. A method for utilizing the ophthalmic device of claim 1, said method comprising:
a) align the tip of eye drop dispenser with the said tip of proximal surface of said nose bridge. This correctly aligns eye drop dispensers of different size with said ophthalmic device.
b) adhere the eye drop dispenser to said top surface of said body of said ophthalmic device by any means for adhering two different objects together.
c) place said nose bridge over top of users nose and slightly tilt user head back.
d) said proximal surface and tip of eye drop dispenser pointed to the eye receiving the eye drop
e) if needed the user may rest their hand onto their face to help stabilize their hand
f) an eye drop is dispensed from the eye drop dispenser onto the medial part of the users eye
g) if the eye drop does not get directly into the users eye, the user can blink or move their head sideways to direct the eye drop into the users eye.
